(19) United States
(12) Patent Application Publication
(10) Pub. No.: US 2015/0010612 A1
(43) Pub. Date: Jan. 8, 2015

(54) BIODEGRADABLE NON-WOVEN MATERIAL FOR MEDICAL PURPOSES

(71) Applicants: HERAEUS MEDICAL GMBH, Wehrheim (DE); CARL FREUDENBERG KG, Weinheim (DE)

(72) Inventors: Sebastian Vogt, Erfurt (DE); Ekaterini Copanaki, Frankfurt (DE); Dirk Grafahrend, Mannheim (DE); Denis Reibel, Herrlisheim (FR); Daniel Neumüller, Weinheim-Lutzel-Sachsen (DE)

(73) Assignees: CARL FREUDENBERG KG, Weinheim (DE); HERAEUS MEDICAL GMBH, Wehrheim (DE)

(21) Appl. No.: 14/375,460

(22) PCT Filed: Jan. 23, 2013
(86) PCT No.: PCT/EP2013/000198

§ 371 Date: Jul. 30, 2014

(30) Foreign Application Priority Data
Feb. 7, 2012 (DE) 10 2012 002 209.3

(51) Int. Cl. A61L 26/00; D01D 10/00

(52) U.S. Cl. CPC A61L 26/0038; A61L 26/0004; A61L 26/0066; D01D 10/00
USPC 424/445; 424/678; 264/8

(57) ABSTRACT

The invention relates to a biodegradable fleece containing (i) at least one polymer for inducing primary haemostasis, (ii) at least one non-proteinogenic, low-molecular, water-soluble activator of secondary haemostasis, and (iii) at least one non-proteinogenic, low-molecular, water-soluble inhibitor of fibrinolysis. The invention also relates to a method for producing a biodegradable fleece, in which (i) a fluidised fiber raw material, and additives if applicable, is placed in a container, (ii) the container is made to rotate, (iii) the fluidised fiber raw material is dispensed from the container by means of centrifugal forces, whereby fibers or filaments are formed, and (iv) a biodegradable fleece is produced from the fibers or filaments. The invention also relates to the use of said biodegradable fleece as a local haemostatic agent.

This application is a 371 of International Patent Application No. PCT/EP2013/000198, filed Jan. 23, 2013, which claims foreign priority benefit under 35 U.S.C. §119 of German Patent Application No. 10 2012 002 209.3 filed Feb. 7, 2012, the disclosures of which are incorporated herein by reference.

The invention relates to a biodegradable fleece, a method for producing said biodegradable fleece as well as the use of said biodegradable fleece as local haemostatic agent.

Surgical interventions are often associated with local haemorrhage in soft tissues that cannot be staunched with common methods of haemostasis, such as direct compression, suturing, clips or cauterisation. Effective haemostasis during surgical interventions can clearly reduce the number of transfusions needed and improve the visualisation of the site of intervention and reduce the surgery time. Moreover, effective haemostasis also reduces the mortality and morbidity of the patients during and after surgical interventions. For this reason, sponges, films, gauze materials and powders made of collagen, cellulose and/or gelatine have been developed for use as local passive haemostatic agents.

It is a disadvantage, especially of the powders, that these often adhere to gloves and instruments of the physicians performing the surgery as a result of electrostatic effects, which makes them difficult to handle. The haemostatic effect of said sponges or films or gauze materials, is based on platelet aggregation at the surface of the proteins or cellulose, of which these are made. This enables the formation of a thrombus and effective closure of the defect. Regarding collagen haemostatic agents, it needs to be taken into account that between 2% and 4% of the total population are allergic to bovine collagen [A. K. Lynn, I. V. Yannas, W. Bonfield; Antigenicity and immunogenicity of collagen; J Biomed Mater Res B Appl Biomater. 2004; 71(2); 343-354]. Cellulose-based products contain regenerated oxidised cellulose. There is some evidence in the literature indicating that these are absorbed less well than collagen and gelatine-based products. Several case studies have shown that it was possible to identify residues of the oxidised cellulose at a revision surgery [Y. Tomizawa; Clinical benefits and risk analysis of topical hemostats: a review; J. Artif. Organs. 2005; 8(3); 137-142]. Due to its properties, gelatine can also be used in the case of irregularly shaped wound geometries. Affixing a haemostatic agent of this type to the site of haemorrhage, the material adapts to the wound and swells which produces a tamponade effect in confined spaces. Swollen gelatine reduces the blood flow and forms a stable matrix about which a thrombus is formed by means of contact activation. Essentially, there are gelatine-based products of bovine and porcine origin available.

A disadvantage in terms of the handling during surgical interventions is the high tackiness of the blood-soaked products on surgical instruments [S. Srinath; Topical hemostatic agents in surgery: A surgeon's perspective; Aorn Journal. 2008; 88(3) 2-11]. Sponges are generally produced by freeze drying and also by special foaming processes. This is disadvantageous since fibroblasts can migrate only with difficulty or not at all into the sponges and swollen powders in the scope of wound healing. In general, the use of excessive quantities of such solely passive haemostatic agents that are based on collagen, gelatine, and in particular cellulose, has been observed to be associated with complications. Residues of the product can cause foreign body reactions, chronic inflammations and/or infections at the site of use, which, in turn, promote the formation of granuloma and prevent optimal healing. Granuloma has been observed at a wide variety of sites with solely passive haemostatic agents [H. E. Achneck, B. Sileshi, R. M. Jamiolkowski, D. A. Albala, M. L. Shapiro, J. H. Lawson; A comprehensive review of topical hemostatic agents: Efficacy and recommendations for use; Annals of Surgery. 2010; 251(2). 217-228].

The process of coagulation is sub-divided into primary haemostasis and secondary haemostasis. The essential step of primary haemostasis is platelet aggregation, which leads to initial closure of the bleeding. Secondary haemostasis is a complex cascading process at the end of which fibrin is released from fibrinogen by the thrombin protease and forms a stable fibrin network through cross-linking Secondary haemostasis can be triggered, inter alia, by calcium ions, i.e. factor IV.

A number of active haemostatic agents based on collagen sponges containing thrombin has been proposed for activating the formation of fibrin from fibrinogen upon contact to blood. Said active haemostatic agents show biological activity and intervene directly in the later phases of the complex cascading process in order to induce a thrombus at the site of haemorrhage. This is to staunch the bleeding rapidly. The presence of fibrinogen in the patient's blood is required for effective haemostasis by means of thrombin, which therefore fails in afibrinogemaemia patients. However, it must be viewed critically, especially regarding the use of human thrombin, that the thrombin needs to be treated appropriately such that any transmission of viruses, such as HIV and HCV, can be safely excluded. Moreover, bovine and human thrombins have been observed to possess a potential to induce antibodies (in up to 94% of the cases) [H. Seyedejad, M. Imani, T. Jamieson, A. M. Seifalian; Topical haemostatic agents; British Journal of Surgery; 2008; 95; 1197-1225]. Although many patients show no clinical anomalies after the development of antibodies, anomalies have indeed been observed in blood coagulation tests, even with fatal outcomes in exceptional cases (anaphylaxis, coagulopathy) [Y. Wai, V. Tsui, Z. Peng, R. Richardson, D. Oreopoulos, S. M. Tarlo; Anaphylaxis from topical bovine thrombin during haemodialysis and evaluation of sensitization among dialysis population; Clin Exp Allergy; 2003; 33; 1730-1734; M. Pope, K. W. Johnston; Anaphylaxis after thrombin injection of a femoral pseudoaneurysm: recommendations for prevention; J Vasc Surg; 2000; 32; 190-191; and K. Tadokoro, T. Ohtoshi, S. Takafuiji, K. Nakajima, S. Suzuki, K. Yamamoto et al.; Topical thrombin-induced IgE-mediated anaphylaxis: RAST analysis and skin-test studies; J Allergy Clin Immunol 1991; 88; 620-629]. In addition, the use of bovine thrombin in the human body has been observed to be associated with severe immune defence reactions.

In the human body, the plasmin protease acts as antagonist of secondary haemostasis. Plasmin cleaves fibrin networks into small fragments. This process called fibrinolysis counteracts secondary haemostasis.

The haemostatic fleeces thus known are disadvantageous in that haemostasis is not attained rapidly and effectively enough in some cases. Specifically, it is a disadvantage of the haemstatic fleeces according to the prior art that the effect of secondary haemostasis is reduced by the body's inherent plasmin protease and that the haemostatic effect of the haemostatic fleeces is thus limited.

It is therefore the object of the invention to provide an improved biodegradable fleece which can preferably be used to overcome the afore-mentioned disadvantages. In particular and preferably, a biodegradable fleece is to be provided that possesses a stronger haemostatic effect than previous haemostatic fleeces. Concurrently, the biodegradable fleece should be easy to use and as inexpensive as possible to manufacture.

Specifically, a fleece is to be developed that activates both primary and secondary haemostasis and in which the fibrin network thus produced is further stabilised. Moreover, the nature of the fleece should be appropriate such that human fibroblasts can migrate into the fleece such that connective tissue can be formed in the course of wound healing. The fleece should, if possible, not contain proteins isolated from human blood in order to sidestep any transmission of infective pathogens, in particular of human viruses.

It is another goal to preferably design the fleece appropriately such that the pH value of the fleece is being stabilised in the physiological neutral pH range such that wound healing cannot be impaired by any shifts in pH.

Moreover, it should be feasible to modify the fleece with anti-infective agents such that local protection of the fleece against microbial colonisation can be attained.

Said objects are solved by providing a biodegradable fleece as described hereinbelow.

Accordingly, the invention provides a biodegradable fleece containing (i) at least one polymer for inducing primary haemostasis, (ii) at least one non-proteinogenic, low-molecular, water-soluble activator of secondary haemostasis, and (iii) at least one non-proteinogenic, low-molecular, water-soluble inhibitor of fibrinolysis.

Moreover, the invention provides a method for producing said biodegradable fleece, whereby (i) a fluidised fiber raw material, and additives if applicable, is placed in a container, (ii) the container is made to rotate, (iii) the fluidised fiber raw material is dispensed from the container by means of centrifugal forces, whereby fibers (1) or filaments (1) are formed, and (iv) a biodegradable fleece is produced from the fibers (1) or filaments (1).

Moreover, the invention provides the use of said biodegradable fleece as local haemostatic agent.

Presently, a biodegradable material shall be understood to mean materials, in particular polymers, and components that degrade and are absorbed under in-vivo conditions. The materials are eliminated via the natural metabolic pathway in this context. This can involve simple filtration processes of the degradation products or proceed after their metabolisation.

The biodegradable fleece contains (i) a polymer for inducing primary haemostasis.

The polymer for inducing primary haemostasis is preferably selected from the group consisting of collagen, gelatine, carboxymethylcellulose, oxycellulose, carboxymethyldextran, and mixtures thereof. These polymers are readily available and are particularly well-suited for building-up the haemostatic fleece and/or the fibers for the haemostatic fleece. The fleece contains at least one non-proteinogenic, low-molecular, water-soluble activator of secondary haemostasis.

The activator of haemostasis is non-proteinogen, if it comprises no α-amino acids, no peptides and no oligopeptides, preferably no peptides at all.

The activator is low-molecular, if its molar mass is less than 1,000 g/mol. Preferably, the activator has a molar mass of less than 800 g/mol, more preferably of less than 500 g/mol, and particularly preferably of less than 200 g/mol.

Preferably, the activator of secondary haemostasis is soluble in water if the solubility of the activator of secondary haemostasis in water at a temperature of 25° C. is at least 100 mg per litre, more preferably at least 500 mg per litre, even more preferably at least 1,000 mg per litre, and particularly preferably at least 2,000 mg per litre.

Said activator of secondary haemostasis preferably acts haemostatic, i.e. it is well-suited to counteract by medical means a bleeding in a patient. Preferably, the activator of secondary haemostasis supports the inherent haemostasis of the body such that the bleeding is staunched more rapidly.

According to a preferred embodiment, the activator of secondary haemostasis is at least one calcium salt. Said at least one calcium salt preferably has a solubility in water of more than 2 g/litre at a temperature of 25° C. Preferably, the at least one calcium salt is selected from the group consisting of calcium chloride, calcium acetate, calcium sulfate dihydrate, calcium lactate, and mixtures thereof. Calcium salts can be used particularly easily in the build-up of a fleece according to the invention. Moreover, they can be converted easily by the organism of the patient.

The fraction of the activator of secondary haemostasis preferably is in the range of 0.1 to 20% by weight, more preferably in the range of 0.5 to 15% by weight, and even more preferably in the range of 1 to 10% by weight, relative to the weight of the fleece.

The fleece contains at least one non-proteinogenic, low-molecular, water-soluble inhibitor of fibrinolysis.

The inhibitor of fibrinolysis is non-proteinogen, if it comprises no α-amino acids, no peptides and no oligopeptides, preferably no peptides at all.

The inhibitor of fibrinolysis is low-molecular, if its molar mass is less than 1,000 g/mol. Preferably, the inhibitor of fibrinolysis has a molar mass of less than 800 g/mol, more preferably of less than 500 g/mol, and particularly preferably of less than 200 g/mol.

Preferably, the inhibitor of fibrinolysis is soluble in water if the solubility of the inhibitor of fibrinolysis in water at a temperature of 25° C. preferably is at least 100 mg per litre, more preferably at least 500 mg per litre, even more preferably at least 1,000 mg per litre, and particularly preferably at least 2,000 mg per litre.

According to a preferred embodiment of the invention, the non-proteinogenic, low-molecular, water soluble inhibitor of fibrinolysis is a lysine analogue. Preferably, the non-proteinogenic, low-molecular, water-soluble inhibitor of fibrinolysis is an amphoteric aminocarboxylic acid. Preferably, the non-proteinogenic, low-molecular, water-soluble inhibitor of fibrinolysis is an α-aminocarboxylic acid. According to a preferred embodiment, the amino group and the carboxyl group of the α-aminocarboxylic acid are separated by at least five carbon atoms and more preferably by exactly five carbon atoms. Preferably, the at least one non-proteinogenic, low-molecular, water-soluble inhibitor of fibrinolysis is selected from the group consisting of 6-aminohexanoic acid, 4-aminomethylbenzoic acid, trans-4-aminomethylcyclohexylcarboxylic acid and mixtures thereof. Said substances have proven to be particularly well-suited for producing the haemostatic fleece and/or for producing the fibers for the haemostatic fleece. Moreover, said substances are non-objectionable for the patient from a medical point of view and can therefore be used in medical devices.

The invention can just as well provide that the amount of the non-proteinogenic, low-molecular, water-soluble inhibitor of fibrinolysis is selected appropriately such same has a pH-stabilising buffering effect at the surface of the biodegradable fleece, whereby the inhibitor preferably buffers the pH value in the range between 6 and 8.

The inhibitor of fibrinolysis having a buffering effect is particularly advantageous because there is then no need to introduce an additional substance as buffer into the biodegradable fleece.

Preferably the fraction of the inhibitor of fibrinolysis is in the range of 0.1 to 20% by weight, more preferably in the range of 0.5 to 15% by weight, and even more preferably in the range of 1 to 10% by weight, relative to the weight of the fleece. With the fractions of the inhibitor of fibrinolysis being as indicated, fibrinolysis is inhibited to a sufficient degree and the pH value is maintained in a preferred range for haemostasis.

According to a preferred embodiment, the fibers of the biodegradable fleece comprise (i) the polymer for inducing primary haemostasis, (ii) the non-proteinogenic, low-molecular, water-soluble activator of secondary haemostasis, and/or (iii) the non-proteinogenic, low-molecular, water-soluble inhibitor of fibrinolysis. It is preferable in this context that (i) the polymer for inducing primary haemostasis, (ii) the non-proteinogenic, low-molecular, water-soluble activator of secondary haemostasis, and/or (iii) the non-proteinogenic, low-molecular, water-soluble inhibitor of fibrinolysis are distributed homogeneously in the fibers of the fleece.

It is particularly preferred to produce the fibers in the presence of the activator of secondary haemostasis and the inhibitor of fibrinolysis. This saves costs during the build-up of the fleece and a homogeneous haemostatic fleece of a simple design is obtained.

Moreover, the invention can provide the haemostatic fleece to comprise at least one anti-infective agent.

Preferably, said at least one anti-infective agent is an antibiotic.

Preferably, said at least one anti-infective agent is present in the fibers of the biodegradable fleece. In this context, said at least one anti-infective agent can just as well be arranged on the fleece surface.

Preferably, said at least one anti-infective agent is soluble in water. Preferably, the at least one anti-infective agent is soluble in water if the solubility of the at least one anti-infective agent in water at a temperature of 25° C. preferably is at least 100 mg per litre, more preferably at least 500 mg per litre, even more preferably at least 1,000 mg per litre, and particularly preferably at least 2,000 mg per litre.

Preferably, a pharmaceutically effective amount of the at least one anti-infective agent is present in the fleece.

It is of particular advantage that the at least one anti-infective agent is coincorporated into the fleece during the production of the biodegradable fleece. For example, the at least one anti-infective agent can be taken up into the fiber material for producing the biodegradable fleece in this context. Said fleeces possess an additional anti-inflammatory effect and counteract an infection of the patient.

The invention can just as well provide the biodegradable fleece to comprise a buffer substance that is poorly soluble in water. Said buffer substance can be present, for example, in the fibers of the fleece and can be distributed homogeneously in the fibers of the fleece, if applicable.

Preferably, a buffer substance is poorly soluble in water if the solubility of the buffer substance in water at a temperature of 25° C. is less than 10 g and more preferably less than 5 g. The solubility of the buffer substance in water at a temperature of 25° C. preferably is in the range of 1 mg/litre to 1 g/litre and more preferably in the range of 5 mg/litre to 500 mg/litre.

According to a preferred embodiment, the buffer substance is selected from the group consisting of calcium carbonate, magnesium carbonate, basic magnesium carbonate, and mixtures thereof.

An additional buffer substance helps in adjusting a suitable pH value for haemostasis in the blood of the patient in the immediate vicinity of the biodegradable fleece inserted into the wound.

Another refinement of the invention can provide the biodegradable fleece to comprise a pH indicator. In this context, the fibers of the fleece, in particular, can comprise a pH indicator.

Said pH indicator preferably has a transition point at a pH of less than pH 7.4. Preferably, the pH indicator is bromocresol purple or bromothymol blue.

The pH indicator can be used to visually check the situation existing at the wound site or whether further treatment measures are required in order to achieve rapid haemostasis.

According to a particularly preferred refinement of the invention, the average mesh width between the fibers of the dry fleece is at least 50 μm. The average mesh width between the fibers of the dry fleece preferably is in the range of 50 μm to 500 μm and more preferably in the range of 100 μm to 200 μm.

A mesh width being within the range specified above allows fibroblasts to grow into the fleece. This effects more rapid wound healing upon the use of a biodegradable fleece according to the invention.

The invention can just as well provide the fibers of the biodegradable fleece to comprise a mean fiber diameter in the range of 0.5 μm to 500 μm, preferably in the range of 2 μm to 300 μm, and more preferably in the range of 5 μm to 200 μm.

Fibers of the diameter specified above are sufficiently strong to prevent individual fibers from breaking and to provide sufficient surface area to the biodegradable fleece in order to dispense sufficient concentrations of the agents.

The biodegradable fleece according to the invention can be used, for example, as local haemostatic agent.

The invention is based, in part, on the surprising finding that combining an activator of secondary haemostasis and an inhibitor of fibrinolysis as water-soluble components of a biodegradable fleece improves the haemostatic effect.

As mentioned above, the human organism has the plasmin protease as an antagonist of secondary haemostasis that cleaves the fibrin network into small fragments.

The process of fibrinolysis therefore counteracts secondary haemostasis. The formation of plasmin is induced by plasminogen activators.

Plasmin can be inhibited by analogues of the amino acid, lysine (K. Aktories, U. Förstermann, W. Forth; Allgemeine und spezielle Pharmakologie und Toxikologie; Elsevier, Urban&Fischer Verlag, 9. edition, 2006; 547). Known for this purpose are, inter alia, α-aminocarboxylic acids, whereby the amino group and the carboxyl group have to be separated by five carbon atoms. Commercially available haemostatic preparations containing the anti-fibrinolytic agent, aprotinin, are associated with significant disadvantages. Bovine aprotinin can trigger anaphylactic reactions [R. N. Kaddoum, E. J. Chidiac, M. M. Zestos, S. D. Rajan, A. Baraka; An anaphylactic reaction after primary exposure to an aprotinin test dose in a child with a severe milk allergy; J. Cardiothorac. Vasc. Anesth.; 2007; 21; 243-244]. Between 0.9% and 2.6% of the patients treated with bovine aprotinin showed hypersensitivity reactions upon repeated exposure [W. Dietrich, A. Ebell, R. Busley, A. Boulesteix; Aprotinin and anaphylaxis: analysis of 12403 exposures to aprotinin in cardiac surgery; Ann. Thorac. Surg.; 2007; 84; 1144-1150].

It has been been found, surprisingly, in the scope of the invention that inhibitors of fibrinolysis, such as lysine analogues, can be used as water-soluble (and thus blood-soluble) components of haemostatic fleeces in order to improve the haemostatic effect of the fleece.

The invention is also, in part, based on a surprising effect, i.e. that lysine analogues, as low-molecular inhibitors of fibrinolysis, incorporated in sufficient amounts into the fleece, buffer the pH value of the surface and immediate vicinity of the fleece to a physiological, nearly neutral pH range.

The prior art includes no local haemostatic material in the form of sponges, wovens or fleeces, which induce primary and secondary haemostasis and concurrently inhibit the fibrinolysis of the fibrin network thus produced.

The invention provides a biodegradable fleece that activates both primary and secondary haemostasis and in which the fibrin network thus produced is further stabilised through inhibition of fibrinolysis.

Moreover, the prior art also includes no local haemostatic material that adjusts the pH value in the immediate vicinity of the haemostatic material in a targeted manner through addition of a neutral range buffer and thus promotes haemostasis through this measure as well.

The pH value of the wound exudate influences the wound healing process. For optimal wound healing, a neutral pH value is of advantage (J. Dissemond; Die Bedeutung des pH-Wertes für die Wundheilung; HARTMANN wundForum 1/2006; 15-19). Therefore, adjusting the pH value to within a desired range according to the invention is also advantageous in the use of fleeces according to the invention. For this purpose, a soluble buffer substance is applied to the fleece. It is particularly advantageous and attains a special additive effect to use the inhibitor of fibrinolysis, which is used anyway, also as buffer substance. For this purpose, it is necessary to simply apply a sufficient amount of the inhibitor to the surface of the fleece such that it has a pH-stabilising buffering effect in the immediate vicinity of the surface of the fleece.

The biodegradable fleece according to the invention can preferably be produced by means of the method for producing a biodegradable fleece as described herein.

In said method (i) a fluidised fiber raw material, and additives if applicable, is placed in a container, (ii) the container is made to rotate, (iii) the fluidised fiber raw material is dispensed from the container by means of centrifugal forces, whereby fibers (1) or filaments (1) are formed, and (iv) a biodegradable fleece is produced from the fibers (1) or filaments (1). Preferably, the container, in which the fluidised fiber material, and additives if applicable, are placed is a spinning rotor.

This production method is particularly easy and inexpensive to implement.

In this context, the invention can provide the fibers and filaments thus produced to be captured as a two-dimensional material upon their exit from the rotating container, whereby connecting sites between two or more fibers are generated in a multitude of regions of the two-dimensional material.

This measure also serves to keep the production method simple and inexpensive.

Moreover, the invention can provide the fleece, in particular the two-dimensional material, to be soaked and/or coated with at least one fluid medium in at least one post-treatment step, whereby, in particular, biologically degradable polymer materials and/or wax-like materials are used as liquid medium.

Biodegradable fleeces according to the invention can be produced in simple and inexpensive manner using a rotation spinning method, for example according to DE 10 2007 011 606 A1, WO 2011/116848, and DE 10 2007 044 648 A1.

The fibers and/or filaments produced by the spinning rotor are easy to capture in a condition, in which connecting sites between two or more fibers are generated in a multitude of regions of the two-dimensional material.

In an optional post-treatment step, a large number of properties of the two-dimensional material according to the invention can be adapted to specific applications.

By cross-linking the material, the mechanical and, in particular, the chemical properties of the biodegradable fleece can be modified. For example, the absorption properties for medical applications can be defined through the degree of cross-linking of the material.

The two-dimensional materials according to the invention can be soaked and/or coated with liquid media in post-treatment steps. For this purpose, in particular but not exclusively, other biologically degradable polymer materials or wax-like materials are conceivable.

The method according to the invention described above can be used to easily produce two-dimensional materials of fibers for biodegradable fleeces according to the invention whose fibers have a mean fiber thickness of 0.5 μm to 500 μm.

For producing partially cross-linked materials in the fibers, it is preferred to add a cross-linker already to the spinning solution. However, the already spun fibers can be cross-linked also and additionally by contacting them to a cross-linker, either in gaseous form or in solution.

According to the invention, finished randomly-oriented mats can be subjected to further cross-linking, which then determines the final degree of cross-linking of the fibers in the two-dimensional material and thus the biological degradation rate thereof.

Various methods are available for cross-linking, whereby enzymatic methods, the use of complexing agents or chemical methods are preferred.

In chemical cross-linking, the cross-linking is performed by means of one or more reactants, in particular using aldehydes selected from formaldehyde and dialdehydes, isocyanates, diisocyanates, carbodiimides, alkyldihalogenides. Moreover, hydrophilic di- and trioxiranes such as, for example, 1,4-butanediol diglycidyl ether, glycerol triglycidyl ether, and polyethylene glycol derivatives can be used. The use of polyethylene glycol diglycidylether is particularly preferred in this context. Aside from cross-linking, polyethylene glycol derivatives showed the beneficial property to prevent undesired adhesions, for example pericardial adhesions in the case of heart surgeries [W. F. Konertz, M. Kostelka, F. W. Mohr et al.; Reducing the incidence and severity of pericardial adhesions with a sprayab le polymeric matrix; Ann. Thorac. Surg.; 2003; 76; 1270-1274].

In particular with regard to medical applications, it is recommended to remove any excess of the cross-linker from the two-dimensional material and/or the randomly-oriented mat after cross-linking

As described above, it is preferred to add a cross-linker already to the spinning solution and to then perform a further cross-linking on the finished two-dimensional material, basically in a second stage, up to the desired degree of cross-linking

The invention can provide the porosity ε of a biodegradable fleece to be given or calculated by the following formula:

$$\begin{matrix}ɛ = 1 - \frac{{ρ}_{Vlies}}{{ρ}_{Bulk}} \\ = 1 - \frac{\frac{{m}_{Vlies}}{{V}_{Vlies}}}{{ρ}_{a} \cdot \frac{{m}_{a}}{( {m}_{a} + {m}_{b} + {m}_{c} )} + {ρ}_{b} \cdot \frac{{m}_{b}}{( {m}_{a} + {m}_{b} + {m}_{c} )} + {ρ}_{c} \cdot \frac{{m}_{c}}{( {m}_{a} + {m}_{b} + {m}_{c} )}}\end{matrix}$$

Here, ρ_{fleece} is the density of the non-compressed biodegradable fleece, ρ_{Bulk} is the density of the fibers of the biodegradable fleece, m_{fleece} is the mass of the biodegradable fleece, V_{fleece} is the volume of the biodegradable fleece, ρₐ is the density of the fiber-forming polymer, ρ_{b} is the density of the activator of secondary haemostasis, ρ_{c} is the density of the inhibitor of fibrinolysis, mₐ is the mass of the fiber-forming polymer in the fleece, m_{b} is the mass of the activator of secondary haemostasis in the fleece, and m_{c} is the mass of the inhibitor of fibrinolysis in the fleece.

If further components are present in the biodegradable fleece, such as, for example, an additional buffer substance or antibiotics, and the porosity of the biodegradable fleece is to be determined, further parameters need to be taken into consideration according to the same pattern as shown above, i.e. the masses (m_{d}, mₑ, . . . ) and densities (ρ_{d}, ρₑ, . . . ) of the additional components.

It is particularly preferred according to the invention that the fiber surface O_{fiber} calculated or given according to the following formula:

$${O}_{Faser} = π \cdot {Ø}_{Faser} \cdot \frac{1 - ɛ \cdot {V}_{Vlies}}{{π ( \frac{{Ø}_{Faser}}{2} )}^{2}} ,$$

whereby Ø_{fiber} is the average diameter of the fibers.

### BRIEF DESCRIPTION OF THE DRAWINGS

Exemplary embodiments of the invention shall be illustrated in the following on the basis of four schematic figures, though without limiting the scope of the invention. The terms, fleece and non-wovens, are used as synonyms in the exemplary embodiments. In the figures:

FIG. 1: shows a gelatine fleece according to the invention with dry anti-microbial substance;

FIG. 2: shows a gelatine fleece according to the invention with wet anti-microbial substance in water;

FIG. 3: shows a gelatine fleece according to the invention with anti-microbial coating; and

FIG. 4: shows a gelatine fleece according to the invention with anti-microbial substance and anti-microbial coating.

### EXEMPLARY EMBODIMENT 1

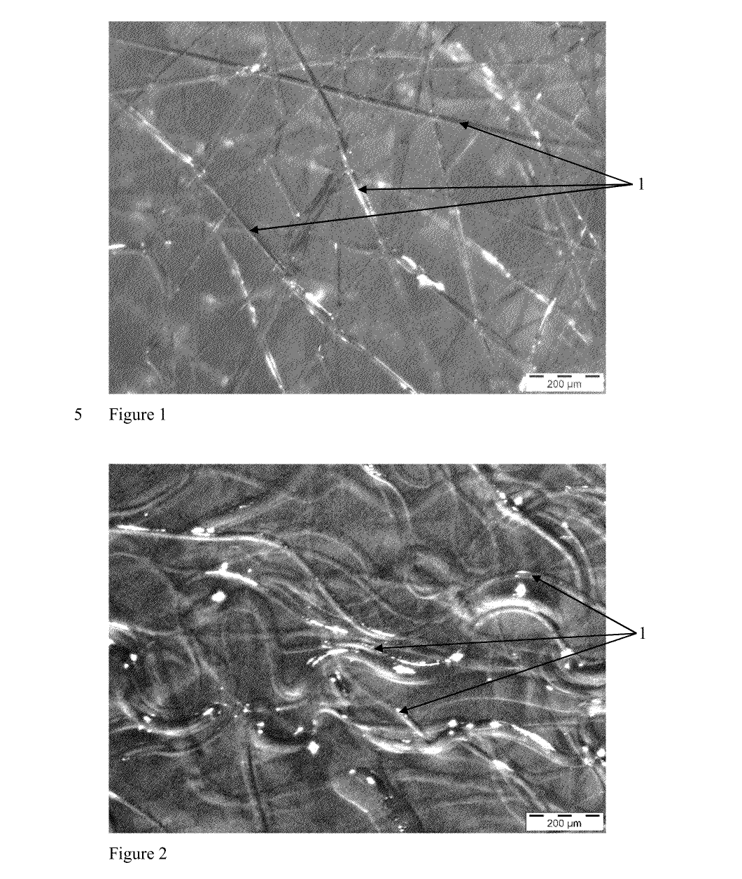

FIGS. 1 and 2 show a first exemplary embodiment of a gelatine fleece according to the invention with dry anti-microbial substance (FIG. 1) and the gelatine fleece according to the invention with wet anti-microbial substance (FIG. 2). The fleece comprises fibers 1 or filaments 1 that are situated as a randomly-oriented mat and are cross-linked to each other. In the wet state according to FIG. 2, the wet fibers 1 are seen to be curved more strongly than in the dry state due to the action of the liquid.

The gelatine fleece with anti-microbial substance according to FIG. 1 (dry) and FIG. 2 (wet; after six hours in distilled water) was produced by means of a rotation spinning method as follows:

Firstly, a 24% gelatine solution was prepared. It is conceivable to use a gelatine of type A PIGSKIN of GELITA AG, which was in fact used in the present exemplary embodiment. The gelatine was stirred in water. The pH was adjusted to 7.4 with NaOH (product number: 3306576, Sigma-Aldrich, Germany). A total of 1% by weight calcium chloride (product number: 102382, Merck, Germany), 1% by weight calcium carbonate (product number: C4830, Sigma-Aldrich, Germany), 1% by weight glycerol (product number: 01873, Sigma-Aldrich, Germany), and 0.5% by weight 6-aminohexanoic acid (product number: 800145, Merck, Germany) were added to the gelatine solution.

The solution was then allowed to stand uninterrupted for approximately one hour to swell. Then, the gelatine solution was treated at 60° C. in an ultrasonic bath and maintained at a temperature of 80° C. to 85° C. for approx. 1 hour. A total of 6% by weight gentamicin sulfate (product number: 345814, Merck, Germany) were then dissolved while stirring in the hot gelatine solution thus produced.

The gelatine solution maintained at 80° C. to 85° C. was guided, as the fiber raw material, by means of a syringe pump into the container of a device for rotation spinning according to DE 10 2005 048 939 A1. A second syringe pump was used concurrently to guide polyethylene glycol diglycidylether (product number: 475696, Sigma-Aldrich, Germany) into the container.

The temperature of the container is approx. 120° C. and the container rotates at a speed of 4,500 rpm. Inside the container there are recesses designed to be holes with a diameter of 0.3 mm. The centrifugal force pressed the fiber raw material through said recesses and spins it into fibers 1 that are drawn by means of an aspiration facility. The aspiration facility was situated below the container.

The fiber diameters were measured using a Zeiss Stemi 2000-C microscope. The mean of 10 single measurements was determined for this purpose.

Fleece samples of 10×10 cm² were used to determine the weight per unit area. The weights were determined using a micro-analytical scale made by Sartorius (model Acculab VIC-123).

The thickness of the fleece samples was determined using a thickness measuring device made by Schroeder (model “Thickness gauge RAINBOW”). In this context, the determination of the thickness must not involve any pressure acting on the fleece to avoid any unintended compression of the fleece and ensuing decrease of the volume.

The porosity ε of the samples was calculated according to the following formula:

$$\begin{matrix}ɛ = 1 - \frac{{ρ}_{Vlies}}{{ρ}_{Bulk}} \\ = 1 - \frac{\frac{{m}_{Vlies}}{{V}_{Vlies}}}{{ρ}_{a} \cdot \frac{{m}_{a}}{( {m}_{a} + {m}_{b} + {m}_{c} )} + {ρ}_{b} \cdot \frac{{m}_{b}}{( {m}_{a} + {m}_{b} + {m}_{c} )} + {ρ}_{c} \cdot \frac{{m}_{c}}{( {m}_{a} + {m}_{b} + {m}_{c} )}}\end{matrix}$$

Here, ρ_{fleece} is the density of the non-compressed biodegradable fleece, ρ_{Bulk} is the density of the fibers 1 of the biodegradable fleece, m_{fleece} is the mass of the biodegradable fleece, V_{fleece} is the volume of the biodegradable fleece, ρₐ is the density of the fiber-forming polymer, ρ_{b} is the density of the activator of secondary haemostasis, ρ_{c} is the density of the inhibitor of fibrinolysis, mₐ is the mass of the fiber-forming polymer in the fleece, m_{b} is the mass of the activator of secondary haemostasis in the fleece, and m_{c} is the mass of the inhibitor of fibrinolysis in the fleece. If further components are present in the biodegradable fleece, such as, for example, an additional buffer substance or antibiotics, and the porosity of the fleece is to be determined, further parameters need to be taken into consideration according to the same pattern as shown above, i.e. the masses (m_{d}, mₑ, . . . ) and densities (ρ_{d}, ρ_{c}, . . . ) of the additional components. The mean pore radius was calculated according to S. J. Eichhorn, W. W. Sampson, Statistical geometry of pores and statistics of porous nanofibrous assemblies, J. R. Soc. Interface, 2005; 2; 309-318. Formula 6.1 on page 315 was used for this purpose.

The fiber surface O_{fiber} was calculated according to the following formula:

$${O}_{Faser} = π \cdot {Ø}_{Faser} \cdot \frac{1 - ɛ \cdot {V}_{Vlies}}{{π ( \frac{{Ø}_{Faser}}{2} )}^{2}} ,$$

whereby Ø_{fiber} is the average diameter of the fibers 1. The contact angle was determined using a goniometer G40 (made by Krüss). For this purpose, one droplet of water was placed on the fleece surface and the wetting angle was measured after 10 s.

Average fiber diameter Ø_{fiber}: 14±5 μm

Weight per unit area: 200 g/m²

Thickness of the samples: 2 mm

Porosity: 0.919

Mean pore radius: 0.1094 mm

Total fiber surface: 462.072 mm²

Contact angle: <30°

### EXEMPLARY EMBODIMENT 2

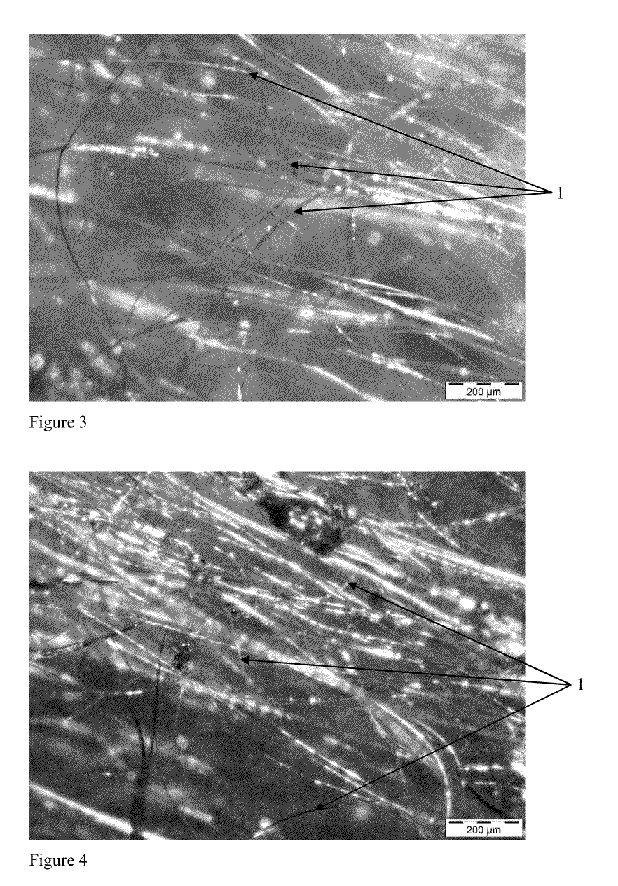

A gelatine fleece with anti-microbial coating according to FIG. 3 was produced by means of a rotation spinning method as follows:

Firstly, a 24% gelatine solution was prepared. A gelatine of type A PIGSKIN made by GELITA AG was used. The gelatine was stirred in water. The pH was adjusted to 7.4 with NaOH (product number: 3306576, Sigma-Aldrich, Germany). A total of 1% by weight calcium chloride (product number: 102382, Merck, Germany), 1% by weight calcium carbonate (product number: C4830, Sigma-Aldrich, Germany), 1% by weight glycerol (product number: 01873, Sigma-Aldrich, Germany), 0.5% by weight trans-4-aminomethylcyclohexylcarboxylic acid (product number: 857653, Sigma-Aldrich, Germany) and 10 mg bromocresol purple (product number: 114375, Sigma-Aldrich, Germany) were added to the gelatine solution.

The solution was then allowed to stand uninterrupted for approximately one hour to swell. Then, the gelatine solution was treated at 60° C. in an ultrasonic bath and then maintained at a temperature of 80° C. to 85° C. for approx. 1 hour.

The gelatine solution maintained at 80° C. to 85° C. was guided, as the fiber raw material, by means of a syringe pump into the container of a device for rotation spinning according to DE 10 2005 048 939 A1. A second syringe pump was used concurrently to guide polyethylene glycol diglycidylether (product number: 475696, Sigma-Aldrich, Germany) into the container.

The temperature of the container is approx. 120° C. and the container rotates at a speed of 4,500 rpm. Inside the container there are recesses designed to be holes with a diameter of 0.3 mm. The centrifugal force pressed the fiber raw material through said recesses and spins it into fibers 1 that are drawn by means of an aspiration facility. The aspiration facility was situated below the container.

The fleece thus obtained was sprayed with a gentamicin palmitate solution (Heraeus Medical, Germany) (5 g dissolved in 100 ml methanol) and dried in a vacuum.

### EXEMPLARY EMBODIMENT 3

A gelatine fleece with anti-microbial substance and antiseptic coating according to FIG. 4 was produced by means of a rotation spinning method as follows:

Firstly, a 24% gelatine solution was prepared. Presently, a gelatine of type A PIGSKIN made by GELITA AG was used, whereby other types of gelatine can be used just as well. The gelatine was stirred in water. The pH was adjusted to 7.4 with NaOH (product number: 3306576, Sigma-Aldrich, Germany). A total of 1% by weight calcium chloride (product number: 102382, Merck, Germany), 5% by weight calcium carbonate (product number: C4830, Sigma-Aldrich, Germany), 1% by weight glycerol (product number: 01873, Sigma-Aldrich, Germany) and 0.5% by weight 6-aminohexanoic acid (product number: 800145, Sigma-Aldrich, Germany) and 10 mg bromocresol purple (product number: 114413, Sigma-Aldrich, Germany) were added to the gelatine solution.

The solution was then allowed to stand uninterrupted for approximately one hour to swell. Then, the gelatine solution was dissolved at 60° C. in an ultrasonic bath and then maintained at a temperature of 80° C. to 85° C. for approx. 1 hour. A total of 6% by weight gentamicin sulfate (product number: 345814, Merck, Germany) were then dissolved while stirring in the hot gelatine solution thus produced.

The gelatine solution maintained at 80° C. to 85° C. was guided, as the fiber raw material, by means of a syringe pump into the container of a device for rotation spinning according to DE 10 2005 048 939 A1.

The temperature of the container is approx. 120° C. and the container rotates at a speed of 4,500 rpm. Inside the container there are recesses designed to be holes with a diameter of 0.3 mm. The centrifugal force pressed the fiber raw material through said recesses and spins it into fibers 1 that are drawn by means of an aspiration facility. The aspiration facility was situated below the container.

The fleece thus obtained was then stored for 12 hours at room temperature in a desiccator in the presence of a 36% formaldehyde solution (product number: F8775, Sigma-Aldrich, Germany) and then evacuated for another 72 hours to fully remove the excess of formaldehyde.

Then, the fleece was sprayed with a polyhexanide solution (Hangzhou Dayang-chem Co., Ltd., China) (5 g polyhexanide in 100 ml of an ethanol/water mixture (80/20; v/v) and dried in a vacuum.

Referring to further advantageous refinements and developments of the teaching according to the invention, reference shall be made to the general part of the description as well as to the appended patent claims. Finally, it shall be noted that the exemplary embodiments have been selected at random and only serve to illustrate the teaching according to the invention without the invention being limited to said exemplary embodiments in any way, manner or shape.

The features of the invention disclosed in the preceding description and in the claims, figures, and exemplary embodiments, can be essential for the implementation of the various embodiments of the invention both alone and in any combination.

### LIST OF REFERENCE NUMBERS

1 Fiber/filament

1. Biodegradable fleece, comprising:
(i) at least one polymer for inducing primary haemostasis;
(ii) at least one non-proteinogenic, low-molecular, water-soluble activator of secondary haemostasis; and
(iii) at least one non-proteinogenic, low-molecular, water-soluble inhibitor of fibrinolysis.

2. Biodegradable fleece according to claim 1, wherein the fleece comprises at least one anti-infective agent.

3. Biodegradable fleece according to claim 1, wherein the fleece comprises fibers (1), whereby the fibers (1) of the fleece comprise (i) the at least one polymer for inducing primary haemostasis, (ii) the non-proteinogenic, low-molecular, water-soluble activator of secondary haemostasis, (iii) the non-proteinogenic, low-molecular, water-soluble inhibitor of fibrinolysis and/or, optionally, at least one anti-infective agent.

4. Biodegradable fleece according to claim 1, wherein the fleece is dry and the average mesh width between the fibers (1) of the dry fleece is at least 50 μm.

5. Biodegradable fleece according to claim 1, wherein the polymer inducing primary haemostasis is selected from the group consisting of collagen, gelatine, carboxymethylcellulose, oxycellulose, carboxymethyldextran, and mixtures thereof

6. Biodegradable fleece according to claim 1, wherein the activator of secondary haemostasis is at least one calcium salt.

7. Biodegradable fleece according to claim 1, wherein the non-proteinogenic, low-molecular, water soluble inhibitor of fibrinolysis is a lysine analogue.

8. Biodegradable fleece according to claim 1, wherein the fraction of the inhibitor of fibrinolysis is in the range of 0.1 to 20% by weight relative to the weight of the fleece.

9. Biodegradable fleece according to claim 1, wherein the fleece comprises a buffer substance that is poorly soluble in water and has a solubility in water at a temperature of 25° C. of less than 10 g.

10. Biodegradable fleece according to claim 1, wherein the fleece comprises a pH indicator.

11. Biodegradable fleece according to claim 1, wherein at least one anti-infective agent is arranged on the surface of the fleece.

12. Biodegradable fleece according to claim 1, wherein the fleece comprises fibers (1) and the fibers (1) of the fleece comprise a mean fiber diameter in the range of 0.5 μm to 500 μm.

13. Biodegradable fleece according to claim 1, wherein the amount of the non-proteinogenic, low-molecular, water-soluble inhibitor of fibrinolysis is selected appropriately such same has a pH-stabilising buffering effect at the surface of the fleece.

14. Method for producing a biodegradable fleece according to claim 1, comprising:
(i) placing a fluidised fiber raw material, and additives if applicable, in a container;
(ii) making the container to rotate;
(iii) dispensing the fluidised fibre raw material from the container by means of centrifugal forces, whereby fibers (1) or filaments (1) are formed; and
(iv) producing a biodegradable fleece from the fibers (1) or filaments (1).

15. Method according to claim 14, which further comprises capturing the fibers (1) and filaments (1) thus produced as a two-dimensional material upon their exit from the rotating container, whereby connecting sites between two or more fibers (1) are generated in a multitude of regions of the two-dimensional material.

16. Method according to claim 14, which further comprises soaking and/or coating the fleece with at least one fluid medium in at least one post-treatment step, whereby, optionally, biologically degradable polymer materials and/or wax-like materials are used as liquid medium.

17. A method of combatting a local haemorrhage in a patient undergoing surgery, said method comprising introducing at the site of the haemorrhage the biodegradable fleece according to claim 1 as a local haemostatic agent.
